(19) United States
(12) Patent Application Publication
(10) Pub. No.: US 2025/0186677 A1
(43) Pub. Date: Jun. 12, 2025

(54) MEDICAL DEVICE AUDIBLE ALERTS

(71) Applicant: ConvaTec Technologies Inc., Las Vegas, NV (US)

(72) Inventors: Peter Strickler, Tewksbury, MA (US); Peter Staats, Beverly, MA (US)

(21) Appl. No.: 19/061,252

(22) Filed: Feb. 24, 2025

Related U.S. Application Data
(63) Continuation of application No. PCT/US23/73333, filed on Sep. 1, 2023.
(60) Provisional application No. 63/403,079, filed on Sep. 1, 2022.

(51) Int. Cl. A61M 1/00; A61F 13/05

(52) U.S. Cl. CPC A61M 1/962; A61F 13/05; A61M 1/84; A61M 2205/18; A61M 2205/3344; A61M 2205/3382

(57) ABSTRACT

A method for controlling a medical device. The method includes providing a conduit coupled to a pump, the pump configured to selectively provide a pressure in the conduit, a motor configured to selectively drive the pump at an operating frequency to provide the pressure in the conduit, and a controller configured to selectively drive the motor and monitor the medical device. The method includes selectively driving the motor at an indicating frequency that is different from the operating frequency when the controller identifies a value outside of a threshold for the medical device. The indicating frequency produces an indicating audible or haptic feedback that is greater than an operating audible or haptic feedback provided at the operating frequency.

### FIELD OF THE DISCLOSURE

The present disclosure relates to providing an audible alert in a medical device having an electric motor, and more particularly to providing an audible alert with a motor and pump used in the medical device.

### BACKGROUND OF THE DISCLOSURE

Negative pressure wound therapy (“NPWT”) devices typically use vacuum pumps to supply negative pressure, and these vacuum pumps can vary in how much audible noise they produce depending on the electrical signals used to drive them. User feedback on NPWT devices is commonly accomplished using LEDs. However, a patient or clinician may look at an NPWT device infrequently. Further, the NPWT device is often worn in inaccessible areas of the body and/or under clothing making a visual inspection of any LEDs difficult. Therefore, in conventional NPWT devices a long period of time may elapse between when a condition requiring intervention occurs and when the visual LED indication is noticed.

### SUMMARY

One embodiment is a negative pressure wound therapy device that has a wound dressing coupled to a pump, the pump configured to selectively provide a negative pressure in the wound dressing, a vacuum pump configured to selectively drive the pump to provide the negative pressure in the wound dressing, and a controller configured to selectively drive the vacuum pump and monitor the wound dressing. The controller selectively drives the vacuum pump in a signal generating cycle when the controller identifies a discrepancy in the negative pressure level.

In one example of this embodiment, the vacuum pump produces an audible signal during the signal generating cycle. In another example of this embodiment, the vacuum pump produces a haptic signal during the signal generating cycle. In yet another example of this embodiment, the controller implements the signal generating cycle when an air leak is detected in the system. In one example, the controller implements the signal generating cycle when the wound dressing is saturated with effluent.

Another embodiment is a method for controlling a medical device. The method includes providing a conduit coupled to a pump, the pump configured to selectively provide a pressure in the conduit, a motor configured to selectively drive the pump at an operating frequency to provide the pressure in the conduit, and a controller configured to selectively drive the motor and monitor the medical device. The method includes selectively driving the motor at an indicating frequency that is different from the operating frequency when the controller identifies a value outside of a threshold for the medical device. The indicating frequency produces an indicating audible or haptic feedback that is greater than an operating audible or haptic feedback provided at the operating frequency.

One example of this embodiment includes operating the motor with the controller at the indicating frequency to produce an audible signal. Another example includes operating the motor with the controller at the indicating frequency to produce a haptic signal.

In yet another example the medical device is a negative pressure wound therapy system that has a wound dressing configured to be positioned over the wound of a user and the conduit is fluidly coupled to a portion of the wound dressing to selectively provide a negative pressure to the wound dressing. Part of this example includes identifying excess effluent in the wound dressing with at least one sensor and implementing the indicating frequency with the controller when the at least one sensor indicates that the wound dressing is saturated with effluent. Another part of this example includes identifying a pressure in the conduit with at least one sensor and implementing the indicating frequency with the controller when the at least one sensor indicates to the controller that the pressure in the conduit is not within a threshold pressure. In yet another part of this example the controller implements the indicating frequency when a leak is detected in the conduit.

In another example of this embodiment, the indicating frequency includes a first pulse pattern associated with a first threshold value and a second pulse pattern associated with a second threshold and the controller selectively operates the motor at the indicating frequency having the first pulse pattern when the controller identifies a first value outside of the first threshold or the second pulse pattern when the controller identifies a second value outside of the second threshold. In part of this example one of the first threshold or the second threshold is a pressure threshold. In another part of this example the other of the first threshold or the second threshold is moisture threshold.

Yet another example of this embodiment includes only driving the motor at the indicating frequency after the controller determines the indicating frequency to be an option based on operating conditions of the medical device.

In one example of this embodiment the motor is a DC motor. In part of this example the indicating frequency is around 100 Hz. In another part of this example the operating frequency is around 27 kHz.

In yet another example of this embodiment the motor is a piezoelectric motor. In part of this example the indicating frequency is between around 100 Hz-4 kHz. In yet another part of this example the operating frequency is around 20 kHz-23 kHz.

### BRIEF DESCRIPTION OF THE FIGURES

The above-mentioned aspects of the present disclosure and the manner of obtaining them will become more apparent and the disclosure itself will be better understood by reference to the following description of the embodiments of the disclosure, taken in conjunction with the accompanying drawings, wherein:

FIG. 1 is a schematic view of a negative pressure wound therapy system; and

FIG. 2 is a schematic view of a logic flow chart.

Corresponding reference numerals indicate corresponding parts throughout the several views.

### DETAILED DESCRIPTION

The embodiments of the present disclosure described below are not exhaustive and do not limit the disclosure to the precise forms in the following detailed description. Rather, the embodiments are chosen and described so that others skilled in the art may appreciate and understand the principles and practices of the present disclosure.

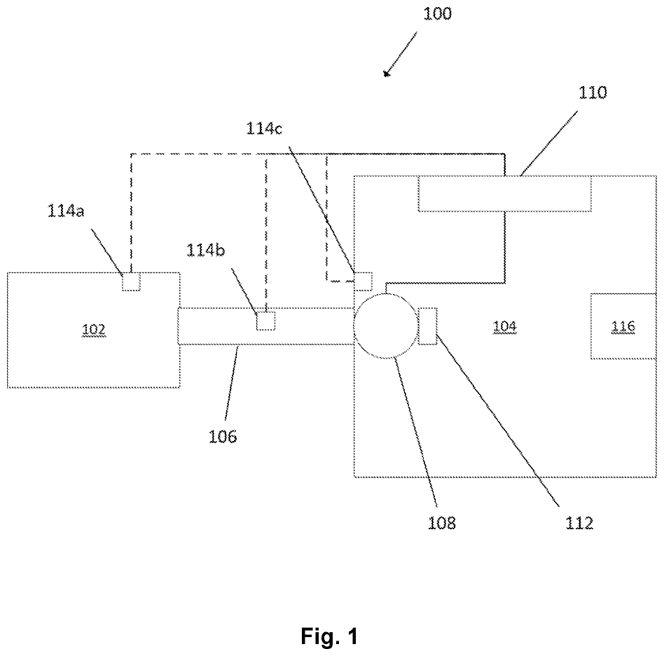

Referring to FIG. 1, a schematic example of a Negative Pressure Wound Therapy (“NPWT”) system 100 is illustrated. While a NPWT system 100 is illustrated herein, this disclosure contemplates applying the teachings discussed herein to any application containing a motor driven pump or the like. Accordingly, the NPWT system 100 is only one example of the many applications considered herein.

The NPWT system 100 may have a wound dressing 102 fluidly coupled to a pump assembly 104 through a conduit 106. The wound dressing 102 may be configured to have a wound-facing side having one or more layers of material to assist in healing an open wound. The wound dressing 102 may further have an outer layer that can be substantially adhered to a user on or around the wound to hold the one or more layers of material against the wound during use. Further, the outer layer may provide an orifice for the conduit 106 to be fluidly coupled to the one or more layers of material such that a vacuum may be applied to the one or more layers of material through the conduit to expedite healing the wound.

The conduit 106 may fluidly couple the wound dressing 102 to a pump 108 in the pump assembly 104. The pump 108 may be configured to provide a vacuum pressure to the wound dressing 102 through the conduit 106. In other embodiments considered herein, a canister may be provided along the conduit to capture excess exudate removed from the wound dressing 102. While specific details are provided herein for the wound dressing 102, pump assembly 104, and conduit 106, this disclosure can be applied to any type of medical device that uses a motor to execute a function. In this particular example of a NPWT system 100, the pump 108 may be selectively powered by a DC or piezoelectric motor 112 to provide a negative pressure to the wound dressing 102.

More specifically, the motor 112 may selectively provide mechanical power to the pump 108 based on input from a controller 110. The controller 110 may selectively power the motor 112, and in turn the pump 108, to selectively control the negative pressure provide to the wound dressing 102. The controller 110 may run the motor 112 at a default and pre-defined speed or alter the operation of the motor 112 based on input received from one or more sensor 114a, 114b, 114c. In one example, a sensor 114a may be positioned on the wound dressing 102 to identify the vacuum pressure or exudate saturation at the wound dressing 102. The controller 110 may use the input from the sensor 114a to alter the operating state of the motor 112 based on the conditions at the wound dressing 102.

Similarly, the sensor 114b may be positioned in, or along, the conduit 106 to identify the operating conditions at the conduit 106 for the controller 110. The operating conditions may include the negative pressure and moisture content at the conduit 106, among other things. The controller 110 may monitor the sensor 114b along with, or instead of, the sensor 114a and modify the operating conditions of the motor 112 and pump 108 in response to operating conditions identified by the sensors 114a, 114b.

The controller 110 may also monitor sensor 114c to identify the operating conditions of the pump assembly 104. In one example, the sensor 114c may be able to identify the operating state of the motor 112 and pump 108. For example, of the motor 112 is a DC motor the sensor 114c may identify the rotational speed of the motor 112 to the controller in revolutions per minute (“RPMs”). Alternatively, if the motor 112 is a piezoelectric motor, the sensor 114c may identify to the controller 110 the frequency by which the motor 112 is vibrating. Further still, the sensor 114c may identify to the controller 110 the operating pressure at the pump 108 among other things.

While specific locations and types of sensors are discussed herein, this disclosure considers applying these teachings to any known NPWT system, among other things, capable of monitoring a system and controlling a motor. Further, the teachings of this disclosure are also applicable to other medical devices having a controlled motor and sensor. Accordingly, the specific sensor locations are one example of this disclosure, but other medical device applications are considered herein as well.

In one aspect of this disclosure, the pump 108 is a vacuum pump and is configured to be selectively controlled with the motor 112 through the controller 110 to generate an audible or haptic signal. More specifically, the motor 112 may drive the pump 108 in a way that maximizes its noise and/or vibration. During a normal pumping operation of the pump assembly 104, one of the objectives may be to minimize noise and avoid disturbing the user. However, as will be explained in more detail herein, the pump assembly 104 may be selectively driven to provide audible and/or haptic feedback to the user. In one example, the controller 110 may selectively drive the motor 112 and pump 108 in a signal generating cycle to produce the audible or haptic signal. In this configuration, the pump assembly 110 can provide a user or healthcare provider with a signal when the NPWT system 100 requires attention. Among other things, the teachings of this disclosure can be implemented without adding substantial additional cost to the build of a NPTWT system 100. More specifically, the pump 108 and motor 112 may already be required to power the pump assembly 104 to provide the negative pressure.

The controller 110 of the NPWT system 100 may be capable of identifying when the NPWT system 100 is not functioning as intended. This may be due to an unintentional leak in the NPWT system 100, when the NPWT system 100 is saturated with effluent, when the NPWT system 100 has reached its maximum recommended lifetime, when the NPWT system 100 has reached the end of its programmed lifetime, when the NPWT system 100 battery is low, when there is a kink in the conduit 106, when the controller 110 detects an NPWT device 100 internal error condition, or other causes. Regardless of the cause, once the controller 110 identifies that the NPWT system 100 is not functioning as intended the pump 108 may be driven by the motor 112 in the signal generating cycle to draw the attention of the user or healthcare provider as an indication that the NPWT system 100 is not functioning as intended.

In one aspect of this disclosure, the controller 110 may identify a leak in the NPWT system 100 by monitoring one or more of the sensors 114a, 114b, 114c. For example, sensor 114a may be a pressure sensor that identifies the pressure provided to the wound dressing 102. If the pressure at the wound dressing 102 is not within an expected pressure threshold based on the operating conditions of the pump 108 and motor 112, the controller 110 may identify an error that indicates a leak in the NPWT system 100.

Similarly, sensor 114b may be a pressure sensor and the controller 110 may monitor the pressure in the conduit 106 through the sensor 114b to check whether the pressure in the conduit 106 is within an expected threshold range based on the operating conditions of the pump 108 and motor 112. If the pressure in the conduit 106 is not within an expected threshold range based on the operating conditions of the pump 108 and motor 112, the controller 110 may identify an error that indicates a leak in the NPWT system 100.

Similarly, sensor 114c may be a pressure sensor and the controller 110 may monitor the pressure at or around the pump 108 through the sensor 114c to check whether the pressure at or around the pump 108 is within an expected threshold range based on the operating conditions of the pump 108 and motor 112. If the pressure in at or around the pump 108 is not within an expected threshold range based on the operating conditions of the pump 108 and motor 112, the controller 110 may identify an error that indicates a leak in the NPWT system 100.

In another aspect of this disclosure, the NPWT system 100 may be powered by an onboard batter 116. The battery 116 may power the controller 110 and be used to selectively power the motor 112 of the pump 108. The battery 116 may be permanent, replaceable, or rechargeable. In one aspect of this disclosure, the battery 116 is permanent and the NPWT system 100 is intended to be entirely discarded after the battery 116 dies. Regardless, in one embodiment contemplated herein the controller 110 monitors the available charge of the battery 116 and selectively provides an indication through the motor 112 and pump 108 when the available power in the battery drops below a threshold. The indication may be understood by the user or healthcare provider to recharge or replace the battery 116 when possible or the indication may be understood by the user or healthcare provider to discard and optionally replace the NPWT system 100 when the battery is permanent.

In yet another aspect of this disclosure, the controller 110 may communicate with one or more of the sensors 114a, 114b, 114c to identify when the dressing 102 and/or conduit 106 are full of effluent. In one example, the sensor 114a is in fluid communication with the wound-facing side of the of the wound dressing 102. When the absorbent layers of the wound dressing 102 are saturated with effluent, the sensor 114a may identify the moisture saturation and indicate the same to the controller 110. When the controller 110 identifies the wound dressing 102 is saturated, it may provide an indication to the user or healthcare provider by selectively running the motor 112 and pump 108 in a corresponding indication protocol.

Similarly, the sensor 114b may be in fluid communication with the conduit 106 flow path between the wound dressing 102 and the pump 108. When effluent or other moisture is absorbed through the wound dressing 102 and progresses towards the pump 108, the sensor 114b may identify the presence of moisture in the conduit to the controller 110. When the controller 110 identifies moisture in the conduit 106, it may provide an indication to the user or healthcare provider by selectively running the motor 112 and pump 108 in a corresponding indication protocol.

In yet another example, the sensor 114c may be in fluid communication with an inlet or outlet of the pump 108. When effluent or other moisture is absorbed through the wound dressing 102 and progresses to the pump 108, the sensor 114c may identify the presence of moisture at the inlet or outlet of the pump 108 to the controller 110. When the controller 110 identifies moisture in the inlet or outlet of the pump 108, the controller 110 may provide an indication to the user or healthcare provider by selectively running the motor 112 and pump 108 in a corresponding indication protocol.

The controller 110 may also track historical data regarding the use of the NPWT system 100. For example, the controller 110 may track the time for which the motor 112 and pump 108 are engaged to provide a vacuum to the NPWT system 100. The controller 110 may also have stored therein a threshold regarding acceptable use time limits for the NPWT system 100. As the engaged time of the motor 112 and pump 108 approaches the acceptable time limits, the controller 110 may provide an indication to the user or healthcare provider that the maximum time threshold is approaching by selectively running the motor 112 and pump 108 in a corresponding indication protocol.

The controller 110 may also use the sensors 114a, 114b, 114c to identify any functional errors with the NPWT system 100. In one example, the sensor 114c may identify when the pump 108 is being powered by the motor 112. In this example, the controller 110 may monitor the sensor 114c to confirm that the pump 108 is functioning as expected when the motor 112 is powered. If the pump 108 is not functioning as expected when the motor 112 is powered, the controller 110 may provide an indication to the user or healthcare provider that the there is a device error by selectively running the motor 112 and pump 108 in a corresponding indication protocol.

Further, the sensor 114c may be coupled to the motor 112 to provide feedback to the controller 110 regarding the operating conditions of the motor 112. For example, the sensor 114c may identify the operational speed of the motor 108 (RPM if the motor 112 is a DC motor and frequency of the motor 112 is a piezoelectric motor). If the operational speed of the motor 112 is not within the expected threshold range, the controller 110 may provide an indication to the user or healthcare provider that there is a device error by selectively running the motor 112 and pump 108 in a corresponding indication protocol.

The NPWT system 100 may also use the motor 112 and pump 108 to provide an indication to the user or healthcare provider that there is a kink or other blockage in the conduit 106. More specifically, the sensors 114a, 114b, 114c may be pressure sensors that communicate the pressures provided from the pump 108, through the conduit 106, and to the wound dressing 102. The controller 110 may compare pressure readings at each sensor 114a, 114b, 114c to determine whether any of the sensors 114a, 114b, 114c are providing offset readings compared to the other sensors 114a, 114b, 114c, indicating a possible kink in the conduit 106. In one example, sensors 114c and 114b may provide a pressure reading to the controller 110 that is within an expected threshold based on the operating conditions of the pump 108 and motor 112. However, sensor 114a may be positioned at the wound dressing 102 and indicating a pressure inconsistent with the upstream sensors 114b, 114c closer to the pump 108. This may be an indication that the conduit 106 is blocked and the controller 110 may provide an indication to the user or healthcare provider to check the conduit 106 by selectively running the motor 112 and pump 108 in a corresponding indication protocol.

The NPWT system 100 may execute any of the indication protocols with the pump 108 and motor 112 discussed herein at the same time as a LEDs or other indicators on the NPTW system 100. This may provide additional likelihood the user or healthcare provider notices the indication protocol to address the issue associated with the indication protocol. For example, the motor 112 of the pump 108 may be a DC motor and normally driven using a PWM signal with a base frequency of 27 kHz to enable inaudible operation of the pump 108 and motor 112 with low vibration. However, the motor 112 might be driven with a PWM signal with a base frequency of 100 Hz to generate audible noise and greater vibration during an indication protocol when the motor 112 is a DC motor. Similarly, motor 112 of the pump 108 may be a piezoelectric blower driven using a resonance frequency greater than 20 kHz to limit audible noise from the pump 108 and motor 112. However, the pump 108 and motor 112 might be driven with frequencies in the 150 Hz to 4 kHz range to generate an audible tone or a sequence of distinct tones during an indication protocol with a piezoelectric motor 112.

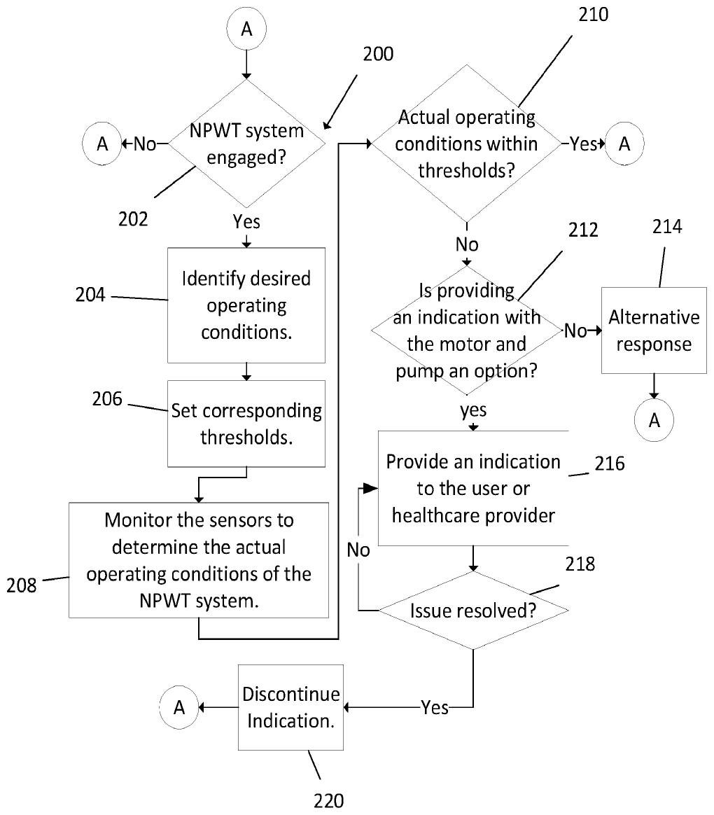

Referring now to FIG. 2, one exemplary logic flow chart 200 is illustrated. The logic flow chart 200 may be an algorithm, computer program, or other stored executable command for the controller 110. Further, the controller 110 may be any known controller capable of executing the logic flow chart 200. The controller 110 may have one or more processor capable of processing commands and otherwise executing the logic flow chart 200. Further, the controller 110 may have, or otherwise have access too, a memory unit capable of storing data and the like. The memory unit may store algorithm for executing the logic flow chart 200 and further may store any thresholds discussed herein. In one aspect of this disclosure, the controller 110 is a circuit board containing the one or more processor and having a memory unit, all stored locally in a housing of the pump assembly 104. Alternatively, the controller 110 may be stored remotely from the pump assembly 104. Regardless, the controller 110 may be a known hardware component capable of storing and executing the flow chart 200 discussed herein, among other things.

The logic flow chart 200 may be initiated by the controller 110 in box 202. In box 202, the controller 110 determines whether the NPWT system 100 has been engaged. The NPWT system 100 may be engaged using any known protocol for such a system. For example, the NPWT system 100 may be engaged by a button that can be selectively touched by the user to turn on or otherwise power the NPWT system 100. Once the controller 110 identifies that the NPWT system 100 is engaged by a user, the controller 110 may identify the desired operating conditions of the NPWT system 100 in box 204. The desired operating conditions can be any known programmable feature of a NPWT system 100. For example, the present disclosure contemplates that the desired operating conditions may include one or more of vacuum pressure, time of applied vacuum pressure, and any other controllable or selectable operating condition of a NPWT system.

Once the controller 110 identifies the desired operating conditions in box 204, the controller 110 may set the corresponding thresholds stored in the memory unit for the desired operating conditions in box 206. In one example, the controller may set the acceptable thresholds for the pressure conditions identified in box 204. Further, the controller 110 may set any other acceptable thresholds for the desired operating conditions in box 206.

Alternatively, the desired operating conditions and corresponding thresholds may be a default values pre-stored into the NPWT system 100. In this configuration, once the NPWT system is engaged in box 202, the default operating conditions and corresponding thresholds will automatically be implemented without requiring user input. Accordingly, other embodiments contemplated by this disclosure do not require boxes 204 and 206 of the logic flow chart 200.

Regardless, once the NPWT system 100 is engaged and the desired operating conditions are identified by the controller 110, the controller may monitor the sensors 114a, 114b, 114c to determine the actual operating conditions of the NPWT system 100 in box 208. As mentioned herein, the sensors 114a, 114b, 114c discussed herein may monitor one or more of pressure, moisture, and operating range of the motor 112 among other things. Accordingly, in box 208 the controller 110 may identify one or more sensor value directed indicating the actual operating conditions of the NPWT system 100.

In box 210, the controller 110 may compare the actual operating conditions identified by one or more of the sensors 114a, 114b, 114c with the thresholds identified based on the desired operating conditions. In other words, in box 210 the controller 110 uses one or more of the sensors 114a, 114b, 114c to identify whether the NPWT system 100 is operating as expected based on the desired operating conditions of box 204. Among other things, the controller 110 may monitor one or more of the sensors 114a, 114b, 114c to determine whether there is a leak based on pressure readings outside of an expected threshold; whether the available battery power is less than an expected threshold; whether the wound dressing 102 is moist and saturated with effluent; whether the use time of the NPWT system 100 is approaching a maximum use time; whether the motor 112 and pump 108 are operating as expected or there is a device error; and whether the conduit 106 is blocked or kinked, among other things.

If the actual operating conditions are within the corresponding thresholds of box 206, the controller 110 may continue to monitor the NPWT system 100 as long as the NPWT system 100 is engaged in box 202. However, if the operating conditions are not within the corresponding thresholds of box 206, the controller 110 may check whether providing an indication with one or more of the motor 112 and pump 108 is an option in box 212. As will be discussed in more detail herein, the controller 110 may provide an indication with one or more of the motor 112 and pump 108 by selectively altering the speed of the motor 112. Accordingly, in box 212 the controller 110 may first check that altering the speed of the motor 112 is an option for providing an indication. As one example, altering the speed of the motor 112 may not be an option if the sensor 114a is identifying an extremely high pressure at the wound dressing 102. Alternatively, in one aspect of this disclosure one of the operating conditions of box 204 may include selecting whether the controller 110 can alter the speed of the motor 112 to provide an indication. If the operating condition was selected to prohibit such a function, box 212 may proceed to an alternative response in box 214.

The alternative response in box 214 may be providing an alert or other warning to a user through an LED or other visual or audible alert system available to the NPWT system 100 that does not require the controller 110 to alter the speed of the motor 112.

If providing an indication with one or more of the motor 112 and pump 108 is an option in box 212, the controller 110 may proceed to provide an indication to the user or healthcare provider by manipulating the speed of the motor 112. For example, if the motor 112 is a brushed or brushless DC motor, the controller 110 may reduce the operating frequency of the motor 112 to provide an audible and haptic feedback to the user. More specifically, the controller 110 may typically drive a motor 112 that is a DC motor at a frequency of around 27 khz to achieve standard vacuum pressures from the pump 108 that are typical for a NPWT system. However, when the controller 110 provides an indication with the motor 112 that is a DC motor the controller may substantially slow the operating frequency to around 100 Hz. The slowed frequency of the DC motor provides an audible tone and noticeable vibrations that may not be apparent to a user or healthcare provider at the operating frequency. Accordingly, the user or healthcare provider will notice a difference in the NPWT system 100 when the controller 110 alters the motor 112 that is a DC motor to provide an indication in box 216.

The provided examples of operating frequencies of a DC motor to provide at least one of an audible and haptic indication non-exhaustive examples, and other operating ranges are also considered herein. More specifically, this disclosure considers operating a DC motor at a substantially reduced speed to provide an indication compared to the intended operating frequency. For example, reducing the frequency of the DC motor allows the user or healthcare provider to hear and feel the movement of the DC motor. In the higher operating frequencies of the DC motor, the user or healthcare provider may not substantially notice when the DC motor is powering the pump 108 during normal operation. However, altering the frequency of the DC motor to provide an indication operates the DC motor to provide an audible and haptic response that is perceivable by one or more of the user and a healthcare provider. Accordingly, the reduced frequency of the DC motor for providing an indication may be any frequency lower than the operating frequency that generates a relatively more perceivable sound and feel from the DC motor than when it is functioning in the intended operating range.

Similarly, if the motor 112 is a piezoelectric motor, the controller 110 may reduce the operating frequency of the motor 112 to provide an audible and haptic feedback to the user. More specifically, the controller 110 may typically drive a motor 112 that is a piezoelectric motor at a frequency of around 20-23 khz to achieve standard vacuum pressures from the pump 108 that are typical for a NPWT system. However, when the controller 110 provides an indication with the motor 112 that is a piezoelectric motor the controller 110 may substantially slow the operating frequency to around 150 Hz to 4 kHz. The slowed frequency of the piezoelectric motor provides an audible tone and noticeable vibrations that may not be apparent to a user or healthcare provider at the operating frequency. Accordingly, the user or healthcare provider will notice a difference in the NPWT system 100 when the controller 110 alters the motor 112 that is a piezoelectric motor to provide an indication in box 216.

The provided examples of operating frequencies of a piezoelectric motor to provide at least one of an audible and haptic indication non-exhaustive examples, and other operating ranges are also considered herein. More specifically, this disclosure considers operating a piezoelectric motor at a substantially reduced speed to provide an indication compared to the intended operating frequency. For example, reducing the frequency of the piezoelectric motor allows the user or healthcare provider to hear and feel the movement of the piezoelectric motor. In the higher operating frequencies of the piezoelectric motor, the user or healthcare provider may not substantially notice when the piezoelectric motor is powering the pump 108 during normal operation. However, altering the frequency of the piezoelectric motor to provide an indication operates the piezoelectric motor to provide an audible and haptic response that is perceivable by one or more of the user and a healthcare provider. Accordingly, the reduced frequency of the piezoelectric motor for providing an indication may be any frequency lower than the operating frequency that generates a relatively more perceivable sound and feel from the piezoelectric motor than when it is functioning in the intended operating range.

In one aspect of this disclosure, there may be many different frequencies for which the motor 112 can be operated to provide different types of indications to a user whether the motor is a DC motor or piezoelectric motor. In one example, if the controller 110 identifies a leak in box 210, the controller 110 may operate the motor 112 in a leak indication frequency. The leak indication frequency may be a frequency that is relatively more perceivable by the user or healthcare provider compared to the operating frequency. Further, the controller 110 may also have a dedicated battery low indicator frequency for the motor 112 that will similarly be relatively more perceivable by the user or healthcare provider compared to the operating frequency but distinguishable from the leak indication frequency. Similarly, additional indicator frequencies may be assigned for the motor 112 to specifically identify which actual threshold of box 210 is not within the threshold values of box 206. That is to say, the controller 110 may operate the motor 112 at a frequency that is relatively more perceivable to the user compared to the operating frequency and specifically associated with a particular indication. In this example, the controller 110 may assign a different perceivable frequency range for the motor 112 for each specific threshold being monitored so the user or healthcare provider can identify both that the NPWT system 100 is not operating within expected thresholds and the specific threshold being missed solely by the altered frequency of the motor 112.

Alternatively, the controller 112 may operate the motor 112 at the lower frequency to provide an indication, but alter the frequency duration to indicate the specific threshold being missed. In this embodiment, the controller 110 may pulse the motor 112 between an indicating frequency, and no frequency, to indicate the specific threshold being missed. For example, if a leak is detected, the controller 110 may pulse the motor 112 at an indicating frequency for one second, followed by stopping power to the motor 112 for one second. This pattern could be executed once or in a continuous loop to indicate to the user or healthcare provider that a leak was detected by based on the sensor value being outside of the threshold. Similarly, a different pattern may be associated with a low battery condition. The pulse pattern implemented by the controller 110 for a low battery may comprise operating the motor 112 at an indicating frequency for three seconds, followed by stopping the motor 112 for a single second. This pattern could be unique compared to that of the leak indication and the at least one user and healthcare provider could associate the specific missed threshold with the patterned indication provided by the controller 110 with the motor 112. Different pulse patterns could be generated for each specific threshold being monitored.

While specific pulse patterns are discussed herein, any discernable pattern is contemplated by this disclosure. The selected pulse patterns for the different indications could be any pulse pattern that could quickly be observed and identified by one or more of the user or healthcare provider.

After, or while, the controller 110 provides an indication in box 216, the controller 110 may continue to monitor the sensors 114a, 114b, 114c to determine whether they indicate values within the operating condition thresholds. If the sensors 114a, 114b, 114c continue to indicate values outside of the operating thresholds in box 218, the controller 110 may continue to provide the indication with the motor 112. However, if the controller 110 identifies that the sensors 114a, 114b, 114c are now showing actual operating conditions within the desired operating conditions, the controller 110 may discontinue operating the motor 112 to provide an indication in box 220 and continue to monitor the NPWT system 100 as discussed herein.

While this disclosure has been described with respect to at least one embodiment, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

1. A method for controlling a medical device, comprising:
providing a conduit coupled to a pump, the pump configured to selectively provide a pressure in the conduit, a motor configured to selectively drive the pump at an operating frequency to provide the pressure in the conduit, and a controller configured to selectively drive the motor and monitor the medical device; and
selectively driving the motor at an indicating frequency that is different from the operating frequency when the controller identifies a value outside of a threshold for the medical device;
wherein the indicating frequency produces an indicating audible or haptic feedback that is greater than an operating audible or haptic feedback provided at the operating frequency.

2. The method of claim 1, further comprising operating the motor with the controller at the indicating frequency to produce an audible signal.

3. The method of claim 1, further comprising operating the motor with the controller at the indicating frequency to produce a haptic signal.

4. The method of claim 1, wherein the medical device comprises a negative pressure wound therapy system comprising a wound dressing configured to be positioned over the wound of a user and the conduit is fluidly coupled to a portion of the wound dressing to selectively provide a negative pressure to the wound dressing.

5. The method of claim 4, further comprising identifying excess effluent in the wound dressing with at least one sensor and implementing the indicating frequency with the controller when the at least one sensor indicates that the wound dressing is saturated with effluent.

6. The method of claim 4, further comprising identifying a pressure in the conduit with at least one sensor and implementing the indicating frequency with the controller when the at least one sensor indicates to the controller that the pressure in the conduit is not within a threshold pressure.

7. The method of claim 4, wherein the controller implements the indicating frequency when a leak is detected in the conduit.

8. The method of claim 1, wherein the indicating frequency comprises a first pulse pattern associated with a first threshold value and a second pulse pattern associated with a second threshold and the controller selectively operates the motor at the indicating frequency having the first pulse pattern when the controller identifies a first value outside of the first threshold or the second pulse pattern when the controller identifies a second value outside of the second threshold.

9. The method of claim 8, wherein one of the first threshold or the second threshold is a pressure threshold.

10. The method of claim 9, wherein the other of the first threshold or the second threshold is moisture threshold.

11. The method of claim 1, further comprising only driving the motor at the indicating frequency after the controller determines the indicating frequency to be an option based on operating conditions of the medical device.

12. The method of claim 1, wherein the motor is a DC motor.

13. The method of claim 11, wherein the indicating frequency is around 100 Hz.

14. The method of claim 13, wherein the operating frequency is around 27 kHz.

15. The method of claim 1, wherein the motor is a piezoelectric motor.

16. The method of claim 11, wherein the indicating frequency is between around 100 Hz-4 kHz.

17. The method of claim 11, wherein the operating frequency is around 20 kHz-23 kHz.
